SEQUENCE LISTING (9 sequences)

SEQ ID NO: 1 - DNA, 23 bp, Artificial sequence
  MNS16A forward primer
aggattctgatctctgaagggtg

SEQ ID NO: 2 - DNA, 22 bp, Artificial sequence
  MNS16A reverse primer
tctgcctgaggaaggacgtatg

SEQ ID NO: 3 - DNA, 20 bp, Artificial sequence
  GAPDH forward primer
gtggacctgacctgccgtct

SEQ ID NO: 4 - DNA, 20 bp, Artificial sequence
  GAPDH reverse primer
ggaggagtgggtgtcgctgt

SEQ ID NO: 5 - DNA, 28 bp, Artificial sequence
  Telomerase substrate sequence
ttttttttttaatccgtcgagcagagtt

SEQ ID NO: 6 - DNA, 34 bp, Artificial sequence
  Human codon-optimised telomerase substrate sequence
ttttttttttaatcccaatcccaatcccaatccc

SEQ ID NO: 7 - DNA, 40 bp, Artificial sequence
  Linker nucleic acid
catgtgtttcgttaggcacctaaggctagctttttttttt

SEQ ID NO: 8 - DNA, 54 bp, Artificial sequence
  Extension nucleic acid
cttaggtgcctaacgaaacacatgaatccgaatccgaatccgaatccgaatccg

SEQ ID NO: 9 - DNA, 24 bp, Artificial sequence
  Extension nucleic acid primer
gaatccacggattgctttgtgtac

(19) United States
(12) Patent Application Publication
(10) Pub. No.: US 2020/0326334 A1
(43) Pub. Date: Oct. 15, 2020

(54) A NANOPARTICLE-BASED TELOMERASE ASSAY

(71) Applicant: University of the Witwatersrand, Johannesburg, Johannesburg (ZA)

(72) Inventors: Boitelo Teresa LETSOLO, Johannesburg (ZA); Martin BERNERT, Johannesburg (ZA); Stefan Franz Thomas WEISS, Johannesburg (ZA)

(21) Appl. No.: 16/772,699

(22) PCT Filed: Dec. 14, 2018
(86) PCT No.: PCT/IB2018/060092

§ 371 Date: Jun. 12, 2020

(30) Foreign Application Priority Data
Dec. 15, 2017 (ZA) 2017/08532

(51) Int. Cl. G01N 33/535; C12Q 1/48; G01N 33/553

(52) U.S. Cl. CPC G01N 33/535; C12Q 1/48; B82Y 5/00; C12Q 2521/113; G01N 2333/9128; G01N 33/553

(57) ABSTRACT

The invention relates to methods for producing gold nanoparticles functionalised with a telomerase substrate, or a linker nucleic acid having a region complementary to a nucleic acid comprising a telomerase substrate, for use in detecting telomerase activity. Functionalised gold nanoparticle solutions and functionalised gold nanoparticles obtained using the method are also provided for. The invention further relates to a functionalised gold nanoparticle complex, comprising gold nanoparticles coupled to a linker nucleic acid which is hybridised to a nucleic acid comprising a telomerase substrate. Telomerase assays using the functionalised gold nanoparticles or functionalised gold nanoparticle complexes of the invention and kits for detecting telomerase activity in a cell, comprising the functionalised gold nanoparticles or functionalised gold nanoparticle complexes of the invention are also provided for.

### BACKGROUND OF THE INVENTION

The present invention relates to a method for producing gold nanoparticles functionalised to detect telomerase activity, comprising combining a gold salt with sodium citrate to produce a gold nanoparticle solution; adding a thiolated nucleic acid to the gold nanoparticle solution; and adding a low pH buffer to obtain a functionalised gold nanoparticle solution, wherein the thiolated nucleic acid is either a thiolated telomerase substrate, or a thiolated linker nucleic acid having a region that is complementary to a nucleic acid comprising a telomerase substrate. The invention further relates to functionalised gold nanoparticle solutions and functionalised gold nanoparticles obtained by the method. The invention also relates to a functionalised gold nanoparticle complex, comprising the gold nanoparticles coupled to a linker nucleic acid which is hybridised to a nucleic acid comprising a telomerase substrate. The present invention further relates to telomerase assays using the functionalised gold nanoparticles or functionalised gold nanoparticle complexes and to kits for detecting telomerase activity in a cell, comprising the functionalised gold nanoparticles or functionalised gold nanoparticle complexes of the invention.

Progressive shortening of the telomeric ends of chromosomes is caused by the semi-conservative mechanism of DNA replication in mitosis. The telomeres are regions of repetitive sequences at the ends of chromosomes. In humans these are 10-15 kbp of TTAGGG DNA repeats. Telomeres have a 3′ overhang which is used to form secondary structures. To form structures such as the telomere loop (t-loop), the 3′ overhang is folded back on itself. These structures in conjunction with other telomere stabilising proteins, including the telosome, a multi-protein structure also known as the “shelterin” complex, help stabilise the ends of chromosomes. The t-loop and the shelterin complex prevent chromosome degradation and fusion, and in doing so regulate telomere length. Thus, the telomeres perform a protective function by preventing the erosion of important coding DNA by the “end-replication” problem. This loss of coding DNA occurs because of the incomplete synthesis of double stranded DNA, which is a characteristic of the mode of action of RNA dependant DNA polymerase.

The enzyme telomerase is a multi-subunit protein that maintains the telomeres by adding a species-dependent telomere repeat sequence to the 3′ end of telomeres to compensate for shortening during replication.

Therefore, telomerase activity serves as a marker of cell proliferation. Telomerase has been shown to be nearly undetectable in most somatic cell lines. Germ-line cells, in addition to other highly proliferating cell types, such as intestinal and oesophageal cells, have been shown to have high telomerase activity. This is most likely due to these cell types requiring constant cell division. If the telomeres are not maintained and a functional DNA damage repair mechanism, such as p53, is still intact, it could lead to replicative senescence or even the induction of apoptosis.

Taking this into consideration, DNA damage response pathways and telomere dynamics can play a vital role in the progression of diseases such as cancer, due to its characteristic uncontrolled cell proliferation. Normally, somatic cells are only capable of undergoing a limited number of cell divisions because of the shortening of telomeres. This is known as the “Hayflick limit”. When approaching this limit, it can lead to the disruption of normal tissue function, which has been theorised to contribute to the aging process. This is further supported by the fact that older individuals have shorter telomeres. This has led to the telomere theory of ageing.

Cancer is characterised by abnormal cell proliferation and is one of the leading causes of death in first world countries and the second leading cause in developing countries. In 2012 alone, over 14 million cases were reported and over 8 million deaths were attributed to cancer worldwide, with sub-Saharan Africa, especially South Africa having one of the highest oesophageal cancer rates in the world.

As telomeres are vital for continued cell proliferation, they play an important role in cancer genetics. Since continued cell division leads to telomere shortening, senescence and even apoptosis may occur as a result. Consequently, senescence acts as a tumour suppressor mechanism, which needs to be circumvented by cells in order for them to become malignant. This means that if cells lack sufficient DNA damage responses, such as in the case of mutated p53, the cells could bypass senescence and become genetically unstable. This second checkpoint is known as “cellular crisis”. “Cellular crisis” is characterised by critically short telomeres and chromosome instability, which can lead to telomere fusions. In order for cancer cells to continue proliferating at this stage, telomeres must either be maintained by the upregulation of telomerase, or through alternative lengthening of telomeres (ALT). ALT is recombination based and is utilised by approximately 10% of cancers, whereas telomerase upregulation can be found in up to 90% or cancers.

Therefore, due to the high number of cancers relying on increased telomerase activity to bypass senescence, telomerase could be a viable target for anti-cancer therapies. In most tumour cells telomerase is activated, thus telomerase activity is an important diagnostic indicator of neoplastic transformation. Consequently, telomerase activity testing is useful for the identification of telomerase inhibitors that have the potential to be anti-cancer drugs as well as for diagnostic purposes.

Further, telomerase activation may also play a role in tissue regeneration, for example, after partial liver resection or cardiac infarction.

In general, telomerase activity detection assays can be divided into two main groups: those based on direct detection of telomerase products, and those based on different systems of amplification of the signals from DNA that yield from telomerase.

To determine telomerase activity in cells, qPCR based telomerase activity assays must be used. Many different telomerase activity assays have been developed, these are often based on the telomeric repeat amplification protocol (TRAP). A TRAP protocol consists of a DNA telomerase substrate which is amplified by the telomerase enzyme in the presence of NTPs. After an extraction process, the enzyme extends the telomeric repeats on the substrate, which can be quantified by a quantitative polymerase chain reaction (qPCR). The number of repeats added is directly proportional to the signal obtained, which is then an accurate measurement of telomerase activity. TRAP based telomerase activity assays are usually reliable, however they can present many different problems. These protocols require whole protein extract from tissues or cells, which can leave impurities behind. Due to the sensitivity of the DNA binding dyes and fluorescent probes utilised by these protocols, these impurities, such as cell debris and genetic material, can adversely affect the experiment. As with most PCR based techniques, non-specific binding and the formation of primer dimers can lead to false positives. TRAP assays can also be very expensive and time consuming and a faster and cheaper alternative would be beneficial.

Direct detection methods conventionally use direct incorporation of radioactively labelled substrate which is then determined electrophoretically. The major drawbacks of this method include the use of large amounts of radioactive isotopes and insufficient sensitivity of the assay.

Metallic nanoparticles, for example gold nanoparticles (AuNPs) have very interesting optical properties. AuNPs are able to change the colour of a nanoparticle colloid solution based on their size and proximity to one another. The larger the AuNPs are in solution, the more the colour of the solution will shift towards blue. On the other hand, the smaller the AuNPs in the solution, the more red shifted the colour of the solution becomes. If AuNPs become aggregated, the colour of the solution also shifts towards blue. This aggregation can be achieved by adding salt to an AuNP solution, which cause the AuNPs to stop repulsing each other and begin to interact with one another. Due to these properties, one could very easily detect changes to the surface of the nanoparticle, as even a small change could result in an observable colour change. This sensitivity as well as their ability to be easily functionalised to many different molecules could make AuNPs a very useful biosensor in detecting telomerase activity.

Detecting telomerase activity using nanoparticles could prove very beneficial, as many steps found in conventional qPCR based methods could be eliminated. This includes the use of fluorescent probes and dyes, which can easily degrade, resulting in differing results from one experiment to another. This would also mitigate signal bleed through in 96-well plates, which can result in false positive results.

### SUMMARY OF THE INVENTION

According to a first aspect of the present invention there is provided for a method of producing gold nanoparticles functionalised to detect telomerase activity, the method comprising:

a) combining a gold salt with sodium citrate to produce a gold nanoparticle solution;

b) adding a thiolated nucleic acid to the gold nanoparticle solution; and

c) adding a buffer having a pH of about 1-4, or about 2-4, to obtain a functionalised gold nanoparticle solution, wherein the gold nanoparticles are coupled to the nucleic acid,

wherein the thiolated nucleic acid is (i) a thiolated telomerase substrate; or (ii) a thiolated linker nucleic acid having a region of complementarity to a nucleic acid comprising a telomerase substrate.

In a first embodiment of the invention the method further comprises centrifuging the functionalised gold nanoparticle solution obtained in step (c) and recovering the functionalised gold nanoparticles. Preferably this centrifugation step is the only centrifugation step. In particular, no centrifugation step is performed between performing step (a) and performing step (b).

In a second embodiment of the present invention the gold salt is chloroauric acid or gold chloride.

In a third embodiment of the present invention the thiolated nucleic acid has a nucleotide sequence corresponding to SEQ ID NO:5, SEQ ID NO:6, or SEQ ID NO:7. In particular, the telomerase substrate nucleic acid has a nucleotide sequence corresponding to SEQ ID NO:5 or SEQ ID NO:6, and the thiolated linker nucleic acid has a nucleotide sequence corresponding to SEQ ID NO:7.

In a further embodiment of the invention the buffer is preferably a sodium citrate buffer, particularly a sodium citrate-hydrochloric acid buffer, having a pH of about 1 or about 2 and the pH of the functionalised gold nanoparticle solution after step (c) is about 2 to 4, preferably the pH of the functionalised gold nanoparticle solution after step (c) is about 3.

According to a further embodiment of the present invention, the method further comprises adding a telomerase substrate having a region of complementarity to the thiolated linker nucleic acid to the solution.

According to a second aspect of the invention there is provided for a functionalised gold nanoparticle solution obtained by the methods described herein.

According to a third aspect of the invention there is provided for a functionalised gold nanoparticle produced by the methods described herein.

In a further embodiment of the present invention the non-functionalised gold particle has a hydrodynamic size of about 40 nm and the functionalised gold nanoparticle has a hydrodynamic size of is about 110 nm in diameter.

According to yet a further aspect of the present invention there is provided for a functionalised gold nanoparticle complex, wherein the complex comprises:

(a) a gold nanoparticle coupled to a linker nucleic acid having a region of complementarity to a nucleic acid comprising a telomerase substrate; and

(b) a nucleic acid comprising a telomerase substrate, wherein the nucleic acid comprising the telomerase substrate is hybridised to the linker nucleic acid to form the functionalised gold nanoparticle complex.

In another embodiment of the invention the linker nucleic acid has a nucleotide sequence corresponding to SEQ ID NO:7 and the nucleic acid comprising the telomerase substrate has a nucleotide sequence corresponding to SEQ ID NO:8.

According to a further aspect of the present invention there is provided for a telomerase assay for detecting telomerase activity in a cell, wherein the assay comprises detecting telomerase activity in the cell using the functionalised gold nanoparticle or the functionalised gold nanoparticle complex described herein.

In yet a further embodiment of the invention wherein the step of detecting in the telomerase assay comprises detecting a colorimetric change, preferably using spectrophotometry, in the cell or in a sample of cells when telomerase is present compared with a reference cell or sample of cells, wherein the reference cell or sample of cells is known to have low telomerase activity. It will be appreciated by those of skill in the art that the colorimetric change is as a result of the extension of the nucleic acid comprising the telomerase substrate by the action of telomerase enzyme in the presence of free nucleotides, which results in steric hindrance between the particles, causing them to disperse.

In one embodiment the telomerase activity is detected in vitro in the cell or sample of cells.

In an alternative embodiment, the telomerase assay may be used to detect telomerase activity in vivo, including detecting telomerase activity in a subject. It will be appreciated by those of skill in the art that the present invention also includes a functionalised gold nanoparticle or the functionalised gold nanoparticle complex for use in a telomerase assay for detecting telomerase activity in a cell, wherein the assay comprises detecting telomerase activity in the cell using the functionalised gold nanoparticle or the functionalised gold nanoparticle complex.

In yet another embodiment of the present invention, the telomerase assay may further comprise a step of assessing telomerase modulating ability of a compound, comprising comparing the telomerase activity in the cell or sample of cells when the compound is present with the telomerase activity in the cell or sample of cells when the compound is not present using the functionalised gold nanoparticle or the functionalised gold nanoparticle complex described herein, thereby identifying a potential telomerase modulator compound or assessing the activity of a telomerase modulator compound.

In a further embodiment of the invention, the cell may be a proliferative cell, preferably a cancer cell, a skin cell, a hair follicle cell, and/or a blood cell.

In yet another aspect of the invention there is provided for a kit including the functionalised gold nanoparticle or the functionalised gold nanoparticle complex described herein or produced by the described methods.

### BRIEF DESCRIPTION OF THE FIGURES

Non-limiting embodiments of the invention will now be described by way of example only and with reference to the following figures:

FIG. 1: Thiolated DNA (telomerase substrate) functionalised AuNPs. After functional telomerase is introduced to the nanoparticle solution, the substrate is elongated. This causes steric hindrance between the particles, causing them to disperse and causes a red shift in colour. The nanoparticle solution with the non-elongated substrate turns a blue colour due to the close association of the nanoparticles.

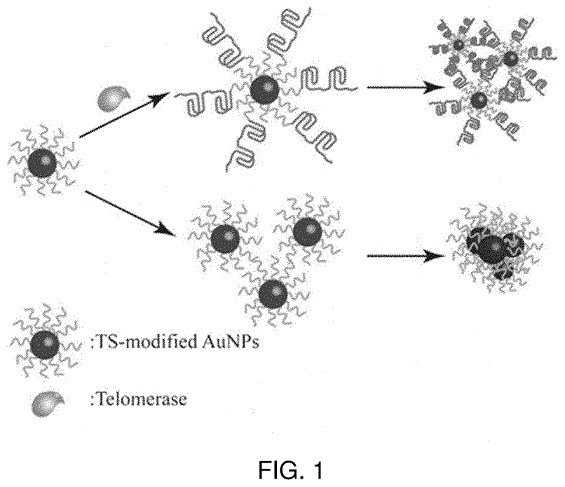

FIG. 2: Effects of metformin on telomerase activity in HEK293, MRC5, SNO, WHCO1 and WHCO5 cells: telomerase activity was determined using qPCR in the HEK293 (panel A), MRC5 (panel B), SNO (panel C) and WHCO1 (panel D) and WHCO5 (panel E) cell lines. Each cell line was treated with 5 and 10 mM metformin for up to 72 h. Statistics were performed at a 95% confidence level (where ***P≤0.001). The MRC5 cell line (B− negative telomerase control) had very low telomerase activity.

FIG. 3: Spectrophotogram of a silver nanoparticle solution: the absorbance of the silver nanoparticle solution was measured in the range of 200-800 nm. The spectrophotogram shows one wide peak at 450 nm which has shoulder at 340 nm. This is indicative of a large size range of the silver nanoparticles.

FIG. 4: Synthesis of silver nanoparticles: SEM and TEM images of the silver nanoparticles were taken. The SEM image (panel A) showed clusters of nanoparticles. However, the TEM image (panel B) showed the presence of nano-rods and nano-triangles in the nanoparticle solution as well as an average spherical particle size of 50 nm.

FIG. 5: Spectrophotogram of a platinum nanoparticle solution: the absorbance of the solution was measured in the range of 200-800 nm. This spectrophotogram showed no distinct peaks.

FIG. 6: Synthesis of platinum nanoparticles: platinum nanoparticles were successfully visualised under SEM (panel A) and TEM (panel B). The particles were clearly visible as large spheres under SEM, however under TEM the particles were very difficult to observe. Under TEM it was seen that the particles were approximately 50 nm in diameter.

FIG. 7: Effects of AuNP size on nanoparticle solution colour: AuNP synthesis yielded very distinct colour differences as the size of the nanoparticles was increased. As the nanoparticles became larger, the solution underwent a blue shift. A gold tinge is also present in the far-left vial.

FIG. 8: Spectrophotogram of a gold nanoparticle solution: the absorbance of the solution was measured in the range of 200-800 nm. A distinct, thin peak can be seen at 530 nm, which may indicate a high degree of uniformity amongst the AuNPs.

FIG. 9: Successful synthesis of AuNPs: the presence of AuNPs was confirmed through SEM (panel A) and TEM (panel B). A greater magnification was not possible in the SEM images, due to the resolution of the microscope as well as the small size of the nanoparticles. Under TEM, the nanoparticles were clearly visible and dispersed. The solution consisted mainly of nano-spheres but few nano-triangles were present. ImageJ analysis of the TEM images confirmed that the average diameter of the AuNPs was 13 nm.

FIG. 10: Protein functionalisation of platinum nanoparticles: platinum nanoparticles were functionalised with lysozyme. The un-modified control (panel A) was very similar to the protein coupled nanoparticles (panel B). However, some particles seemed to have a coating which may be evidence of the protein attaching to the nanoparticles.

FIG. 11: Effects of DNA functionalisation on AuNP spectrophotogram and solution colour: the spectrophotogram of the modified and unmodified nanoparticles (panel A), where black indicates the control and red indicates the DNA coupled AuNPs, shows a distinct shift in the single peak from 530 nm to 550 nm as well as a lowering of the peak. The red graph also has an additional peak at 260 nm which represents the DNA. Panel B shows the colour change that occurred after functionalisation.

FIG. 12: TEM analysis of DNA functionalised AuNPs: TEM images were taken of the functionalised (panel B) and non-functionalised (panel A) AuNPs. A clear halo was seen around DNA modified AuNPs (panel B), which could not be seen in the unmodified control (panel A). (Larger AuNPs, approximately 50 nm in diameter were used to show the halo, as the larger halo was more easily distinguishable).

FIG. 13: Zetasizer analysis of AuNP size distribution using light scattering: Zetasizer measurements were taken on the AuNP solution before and after DNA functionalisation. The height of the peak signifies the amount of the standard size of the particles and the width of the peak shows the size deviation from the standard. The unmodified AuNP solution (panel A) shows a single peak at 40 nm and panel B shows the results of the DNA modified AuNPs, where two distinct peaks, one at 40 nm and one at 110 nm can be seen. Panel C shows the results of DNA modified AuNPs after the addition of the HEK293 protein extract. The two peak sizes (at 40 nm and 110 nm) are still present, however there is evidence of massive aggregation (peak from 1000 nm to 6000 nm).

FIG. 14: Modified AuNP based telomerase activity assay colour change: shows the colour change of the nanoparticle solution after performing the AuNP assay. Tube A contains unmodified AuNPs as well as the reaction buffer necessary for the telomere extension reaction and has a red colour. Tube B contains DNA modified AuNPs as well as the reaction buffer and has a light purple colour. Tube C contains the DNA modified AuNPs, reaction buffer and protein extract, where aggregation can easily be seen at the bottom of the tube. Tube D contains the same components as tube C, however the extension reaction has been allowed to proceed. This sample has a light blue colour and very little aggregation is present.

FIG. 15: TEM analysis of the modified AuNP based telomerase activity assay: shows unmodified AuNPs (panel A), which are largely dispersed. The DNA functionalised AuNPs (panel B) show increased association with one another. Panel C shows DNA modified AuNPs in the presence of a protein extract. Here high amounts of aggregation can be seen. Panel D on the other hand, shows the same sample as seen in panel C, however the telomere extension reaction was performed. This sample still shows signs of aggregation, but far less that that seen in panel C.

FIG. 16: Relative telomerase activity of the AuNP based telomerase activity assay in SNO and WHCO5 cells: after the telomere extension reaction, the absorbance of the nanoparticle solutions containing protein extracts from metformin treated SNO (panel A) and WHCO5 (panel B) cell lines was obtained. No significant difference could be seen between treated and untreated samples within the cell lines.

FIG. 17: The UV-Vis normalised spectra (400 nm-700 nm) for three separate batches of AuNP solutions.

FIG. 18: Graph showing the ratio of the absorbance at two wavelengths (610 nm and 520 nm) of three batches of AuNPs.

FIG. 19: TEM image of AuNPs: Panel A shows a TEM image of Batch 1; Panel B shows a TEM image of Batch 2; and Panel C shows a TEM image of Batch 3.

FIG. 20: A graph showing the size distribution of the AuNPs. The diameter of the spherical nanoparticles was obtained using ImageJ.

FIG. 21: Thiol-DNA functionalised AuNP spectra before and after the addition of the Thiol-DNA to the surface of the AuNPs. The unmodified AuNPs have a peak at 520 nm and the linker DNA functionalised AuNPs have a peak at 650 nm as well as a smaller peak at 520 nm, indicating that most of the AuNPs were surface modified with the linker nucleic acid.

FIG. 22: Graph showing the ratio of the absorbance at two wavelengths (610 nm and 520 nm) of both unfunctionalised AuNPs and AuNPs functionalised with the linker nucleic acid.

FIG. 23: TEM image of linker DNA functionalised AuNPs: Panel A—unfunctionalised AuNPs; and Panel B—linker DNA functionalised AuNPs. Panel B shows a distinct halo around many of the particles indicating the presence of DNA on the surface of the AuNPs.

FIG. 24: Graph of the absorbance ratio (610 nm/520 nm) over time. The HEK293 telomerase positive control showed higher ratio than that of the heat-treated (HEK293 HT—telomerase inactivated) control.

FIG. 25: Diagram of the linker functionalised AuNP telomerase activity assay. A gold nanoparticle is coupled to a 3′ thiolated linker nucleic acid strand which contains a complimentary sequence for an extension nucleic acid strand. The extension nucleic acid strand contains a binding site for telomerase and will be elongated by telomerase.

### SEQUENCE LISTING

The nucleic acid and amino acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:

SEQ ID NO:1—Nucleic acid sequence of MNS16A forward primer.

SEQ ID NO:2—Nucleic acid sequence of MNS16A reverse primer.

SEQ ID NO:3—Nucleic acid sequence of GAPDH forward primer.

SEQ ID NO:4—Nucleic acid sequence of GAPDH reverse primer.

SEQ ID NO:5—Nucleic acid sequence of the telomerase substrate.

SEQ ID NO:6—Nucleic acid sequence of the human codon-optimised telomerase substrate.

SEQ ID NO:7—Nucleic acid sequence of the linker nucleic acid.

SEQ ID NO:8—Nucleic acid sequence of the extension nucleic acid.

SEQ ID NO:9—Nucleic acid sequence of the extension nucleic acid PCR primer.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

The present invention relates to the production of gold nanoparticles (AuNPs) functionalised with DNA. Specifically, the present invention provides for a method of synthesising and functionalising AuNPs with thiolated telomerase substrate or thiolated linker nucleic acids that complementarily bind a nucleic acid that comprises a telomerase substrate. The functionalised particles of the invention have been characterised using transmission electron microscopy and are useful for assessing telomerase activity. To assess telomerase activity the extracted protein from cells suspected of having telomerase activity was added to the functionalised nanoparticle solution and allowed to elongate the coupled DNA. A characteristic of gold nanoparticles is that the size of the particles as well as their proximity to one another determines the colour of the nanoparticle solution. Due to the steric hindrance caused by the elongated DNA, a distinct colour change was observable. The change in absorption spectra of the nanoparticle solution was recorded after the enzyme elongated the substrate. This nanoparticle based assay was then compared to TRAPeze RT Telomerase detection kit (Merck-Millipore) as a positive control. A colour change was observed with the nanoparticle assay compared to the negative control reflecting detection of telomerase activity.

This technique of measuring telomerase using functionalised gold nanoparticles has great potential, as nanoparticle based assays are known for their high sensitivity. The assay of the present invention is also far more rapid and significantly cheaper to produce than the commercially available that the qPCR based assay. The gold nanoparticle based telomerase activity assay could become an alternative to conventional qPCR based techniques. In conventional DNA functionalisation methods using NaCl salt ageing (where the concentration of NaCl is increased over many hours) to functionalise the DNA to the nanoparticles, the nanoparticles first need to be purified by successive centrifugation steps to remove the salt in order to use the modified nanoparticles for downstream applications. This poses a problem, as this may result in the loss of nanoparticles, as well as possibly leading to unrecoverable nanoparticle aggregation. Conventional functionalization techniques are also often labour intensive and very time consuming, some taking up to two days with constant monitoring (as in the salt ageing method).

The pH dependant method of the present invention relies on the interaction of the thiol-modified DNA with the AuNPs by decreasing the pH of the solution. The pH of the solution is reduced from pH7 to pH2-4 using a sodium citrate-hydrochloric acid buffer (pH2). The method of the present invention is convenient, as it requires no additional purification steps after synthesis due to the use of sodium citrate as the reducing agent. This may significantly increase the speed at which the nanoparticles become functionalised as the reactions should take place as soon as the low pH is achieved. Further, the problem of nanoparticle aggregation is overcome by removing the need for successive centrifugation steps and a higher concentration of functionalised gold nanoparticles in the solution is achieved.

Nanoparticle aggregation and thus increased functionality for use in a telomerase activity assay is also overcome by using the linker-functionalised AuNPs of the present invention. Further, the linker-functionalised AuNPs are likely to be more stable. The linker nucleic acid-functionalised AuNPs of the present invention are also useful for further downstream applications, for example quantitative single telomere length-like analysis. The AuNPs are coupled to a 3′ thiolated linker nucleic acid strand which contains a complimentary sequence for an extension strand. The extension strand includes a binding site for telomerase, thus the extension nucleic acid strand will be elongated by telomerase. This elongation can be detected using UV-Vis spectrophotometry. The linker-functionalised AuNPs also have the potential to be reused, as the extension strand can be decoupled from the linker strand by simple thermal denaturation.

Telomerase assays have use in detecting cells that have increased telomerase activity for diagnosing cancers and for identifying candidate drugs that modulate telomerase activity.

The terms “nucleic acid”, “nucleic acid molecule”, “oligonucleotide” or “polynucleotide” encompass both ribonucleotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides.

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation).

A “cell”, “cell sample”, “sample”, “cell extract” or “tissue extract” refers to a cell or biological extract obtained from cells or tissues for which telomerase activity is being tested. In mammals, such cells may be selected from hair follicle cells, peripheral blood cells, cancer cells, buccal cells, skin cells or any other cells from the subject. A cell may also be an in vivo cell.

As used herein the term “subject” refers to a mammalian subject, in particular a human subject.

The term “complementary” refers to two nucleic acids molecules, e.g., DNA or RNA, which are capable of forming Watson-Crick base pairs to produce a region of double-strandedness or complementarity between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the activity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as at least about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

The term “telomerase substrate” refers to is an oligonucleotide chosen to be recognized by the mammalian telomerase to be assays. If one is using the present method to determine the level of telomerase activity in a human subject, one employs a telomerase substrate recognized by human telomerase reverse transcriptase, preferably a codon-optimised human telomerase substrate. The telomerase substrate of the present invention may also be used to determine the level of telomerase activity in a mammalian cell other than a human and is preferably codon-optimised for the mammalian subject for which telomerase is being assayed.

The term “telomerase activity” refers to the activity of a telomerase reverse transcriptase protein in the presence of a telomerase substrate. In particular, the activity of the telomerase is the addition of telomeric DNA repeats to a telomerase substrate per unit time.

A “telomerase modulator” is a compound that directly or indirectly either inhibits or activates the expression or activity of telomerase. A “telomerase modulator” may be a “telomerase inhibitor” or a ‘telomerase activator”.

The term “cancer” refers to the physiological condition in an individual that is typically characterized by unregulated cell growth. Examples of cancers include, but are not limited to, carcinoma, lymphoma, blastoma, sarcoma, and leukaemia. More particularly, examples of such cancers include bone cancer, blood cancer, lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the oesophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumours, glioma, meningioma, and pituitary adenoma.

The examples herein are based on a telomerase activity assay for determining the effect of metformin on telomerase activity in oesophageal cancer cell lines, however it will be appreciated by a person of skill in the art that the invention described herein is not limited to oesophageal cancer cell lines, nor to detecting the effect of metformin on telomere activity.

The following examples are offered by way of illustration and not by way of limitation.

### Example 1

Cell Culture

Cell Culture Protocol

Oesophageal carcinoma cell lines; WHCO1, WHCO5 (Veale and Thornley, 1989), lung fibroblast; MRC5 (Jacobs et al., 1970), SNO (Bey et al., 1976), and human embryonic kidney cells, HEK293 (Graham et al., 1977), were cultured. HEK293 cells were used to optimise procedures involving telomerase activity, as these cells are known to display relatively high levels of telomerase activity. The MRC5 cell line was used as negative control as it expresses very little to no telomerase activity. Ethics approval was obtained from the Human Research Ethics Committee (Medical): reference number W-CJ-140804-1.

Each of the five cell lines were cultured in 88% Dulbecco's Modified Eagle's Medium (DMEM) containing 10% Foetal Calf Serum, 1% Penicillin-Streptomycin and 1% L-Glutamine. The cells were kept in a 5% CO₂, 37° C. humidified atmosphere to ensure that the pH of the system remained constant through the CO₂/HCO₃-buffering system and that the cells experienced maximum cell growth.

Each cell culture flask was viewed under an inverted light microscope to monitor cell growth, cell detachment and to determine the level of confluency. After the cells reached approximately 80% confluency, they were harvested and passaged to prevent contact inhibition and therefore allow the cells to continue growing. To passage the cells, the cells were first detached from the flask through trypsin-EDTA treatment. The trypsin-EDTA was then inactivated by adding an equal volume of cell culture medium. The cells were then evenly passaged into multiple culture flasks. The cells were treated with metformin while the untreated controls were cultured alongside the treated cells.

After harvesting, some cells were cryopreserved to be used at a later stage. After trypsinisation the cells were harvested and centrifuged at low RPM (±200-400 g) for 10 minutes. The excess media was then removed and the pellet resuspended in a cryopreserving solution (15% Glycerol, 20% FCS, 65% DMEM). The vials were then kept at −20° C. overnight and then transferred to −80° C. or liquid nitrogen storage. When thawing the frozen cells, an initial high FCS concentration of 20% was used to provide extra nutrients for the cells. This was then stepped down to 10% once the cells stabilised.

Cell Quantification

To accurately quantify the number of cells in each flask once they reached confluency, a Neubauer haemocytometer was used. The cells needed to be quantified to ensure subsequent experiments and subcultures utilised consistent numbers of cells for reliable results. To distinguish between dead and live cells, the trypan blue stain was used. This stain is only taken up by dead cells, and is excluded by live cells, which leads to dead cells being stained blue under the microscope. This allows for easy quantification of unstained, live cells. The stained suspension of cells was diluted and a small sample added to the Neubauer haemocytometer. Live cells were then quantified, using the 16-square region of the haemocytometer, while utilising a light microscope. Thereafter, cell concentration, total number of cells and cell viability were calculated using the following formulas:

$$Total number of cells = Cell concentration ( \frac{cells}{ml} ) \times total volume of suspension$$

$$Cell concentration ( \frac{cells}{ml} ) = cells per 16 squares \times {10}^{4} \times dilution factor$$

$$Cell viability ( \% ) = ( \frac{number of viable cells}{total number of cells} ) \times 100$$

MTT Cell Viability Assay

The MTT (3-(4,5-dimethylthiazol-2-yl)-2,5-diphenyltetrazolium bromide) assay was used to determine cell viability. As the metformin treatment may affect cell proliferation and cell viability, this assay is very important to perform. The yellow MTT reagent is added to a plate of cells. Viable cells then reduce the reagent through cellular NAD(P)H-dependent oxido-reductases. This turns the MTT reagent into an insoluble formazan product, which is purple in colour, for 4 hours. The insoluble precipitate is then treated with a detergent and mixed until fully dissolved. After the formazan crystals are dissolved, the solution is read at 600 nm, using a spectrophotometer. The higher the amount of dissolved product, the higher the absorbance reading and therefore the more viable cells are present. This assay was performed in 96-well plates, where each well of a 96-well plate was seeded with 5000 cells. They were allowed to attach overnight and treatment commenced the following day. Each treatment as well as each control was done in triplicate. The controls included the no-cell control, no-MTT control, 100% dead cells (treated with 1% triton-x) as well as the untreated cell control. After the addition of the MTT reagent, the colour change was visualised using DMSO and an ELISA reader. The absorbance readings of the treated samples were then compared to those of the controls.

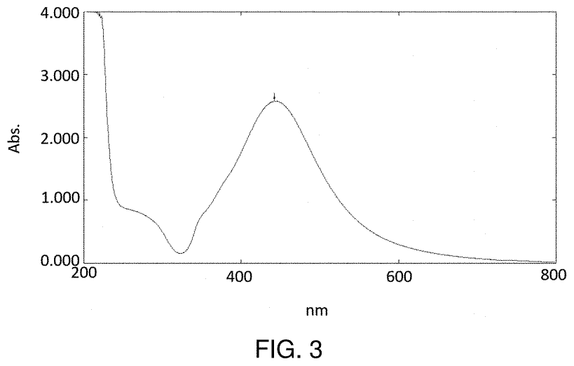

HEK293, MRC5, SNO, WHCO1 and WHCO5 cell lines were successfully cultured and treated with 5 and 10 mM metformin for up to 72 h. Increased cell detachment was observed at these metformin concentrations after 48 and 72 h, compared to the untreated controls which had little to no detachment. The MTT assay was performed on each cell line to determine if metformin affected cell viability. Equal numbers of cells (5000) were added into each well of a 96-well plate. The cell lines were treated for 48 and 72 h with 5 and 10 mM metformin. No significant difference in cell viability was seen for the HEK293, MRC5, SNO and WHCO1 cell lines (FIG. 3). The WHCO5 cell line however showed a significant reduction in cell viability, compared to the untreated control, for both the 48 h and 72 h treatments.

Nucleic Acid Extraction

DNA and RNA were extracted from the cells using a modified phenol chloroform method as well as the Quick-RNA MiniPrep Kit (Zymo Research) respectively. In each case the cells were harvested and washed with PBS prior to pelleting them by centrifugation at 10000 g. The pellets were then washed with PBS to ensure no media remained.

DNA Extraction

A modified phenol chloroform extraction method was used to extract DNA from the various cell lines. The extraction buffer was made up of tris, glycerol, SDS and mercaptoethanol. This buffer was used to lyse the cells. Proteinase K was then added to digest the protein. To separate the cell debris and protein from the lysate containing the DNA, isopropanol and phenol chloroform were added to the cell lysate. The solution was then centrifuged at 10000 g for 5 min at 4° C. to pellet the cell debris, leaving the protein in the organic layer. Whilst the aqueous solution was then transferred to a new tube and mixed with ethanol. The solution was then centrifuged at 10000 g for 5 min at 4° C. again in order to precipitate the genomic DNA. Ethanol is then removed by air-drying the DNA pellet prior to resuspending in TE buffer. Both the amount as well as the purity of the DNA was then determined using the Thermo Scientific ND1000 NanoDrop spectrophotometer. After this, the DNA was resolved on a 1% agarose gel to determine integrity.

Polymerase Chain Reaction (PCR) and Agarose Gel Electrophoresis

To assess the suitability of the DNA for PCR, MNS16A minisatellite as well as GAPDH and were amplified in all cell lines. Table 1 shows the PCT primers that were used.

TABLE 1
PCR Primers

| Primer | 5′-3′ Sequence | Region | SEQ ID NO |
|---|---|---|---|
| MNS16A | F-AGGATTCTGATCT | MNS16A | SEQ ID NO: 1 |
| | CTGAAGGGTG | | |
| | R-TCTGCCTGAGGAA | Mini- | SEQ ID NO: 2 |
| | GGACGTATG | satellite | |
| GAPDH | F-GTGGACCTGACCT | GAPDH | SEQ ID NO: 3 |
| | GCCGTCT | | |
| | R-GGAGGAGTGGGTG | | SEQ ID NO: 4 |
| | TCGCTGT | | |

For all PCR amplifications, KapaTaq Master Mix (Lasec) was used. This PCR master mix contains all the components needed for amplification, such as dNTPs, reaction buffer, magnesium chloride and taq-polymerase. The primers were added separately (Table 1). All amplifications were performed in a MJ Mini thermal cycler (BioRad) using the following thermal cycle: Initial denaturation was performed at 95° C. for 2 minutes, followed by 35 cycles of denaturation (95° C. for 30 seconds), annealing (55° C. for GAPDH and 56° C. for the minisatellite for 30 seconds) and elongation (72° C. for 1 minute). A final elongation step was also performed at 72° C. for 2 minutes to ensure that all reactions had completed.

After the PCR amplification, samples were resolved on agarose gels, using GRGreen (Inqaba Biotech) as a nucleic acid stain. GRGreen intercalates into DNA and creates a detectable fluoresces signal under UV light. The GAPDH sequence and the MNS16A minisatellite products were resolved on a 2.5% agarose gel. The gels were resolved at 80 V for 45 minutes to separate the DNA fragments by size. A DNA weight marker of known fragment lengths was used in order to determine the size of the amplified samples. The gels were then visualised under UV light.

Data Analysis and Statistical Evaluation

The data collected from these experiments were analysed and evaluated in Microsoft Excel and GraphPad Prism (v6.05). Normalising as well as sorting in the obtained data was done in Microsoft Excel, whereas the statistical analysis was performed in GraphPad Prism, using ordinary one-way ANOVA at 95% confidence level between all sample groups.

### Example 2

Quantitative Polymerase Chain Reaction (qPCR) Telomerase Activity Assay

Telomerase Activity

The TRAPeze RT Telomerase Detection Kit (Merck-Millipore) was used to determine telomerase activity for all cell lines. qPCR amplifies a target DNA strand like conventional PCR, but is also able to simultaneously quantify the synthesis of the DNA strand. This is achieved using a sequence specific primer/DNA probe containing a fluorescent reporter as well as a quencher molecule. These reporter molecules fluoresce; however, due to the proximity of the quencher molecule, the fluorescent signal is suppressed. As the telomerase substrate is elongated by the telomerase enzyme, extracted from the cell lines, the probe causes a complimentary strand to be synthesised by taq polymerase. The incorporation of the probe causes the distance between the quencher and fluorophore to increase, creating a fluorescent signal. Therefore, the fluorescent signal is proportional to the amount of added telomeric repeats. Protein was extracted from cell pellets using 3-[(3-Cholamidopropyl)dimethylammonio]-1-propanesulfonate (CHAPS) lysis buffer and the amount of protein being used was standardised to 0.2 mg/ml obtain reliable data (final concentration of 20 ng/μl). The HEK293 cells were used as a positive control for telomerase activity. The following thermal cycle was used: Pre-incubation consisted of one cycle of 37° C. for 30 minutes and 95° C. for 2 minutes. Amplification consisted of 45 cycles of 95° C. for 30 seconds, 59° C. for 1 minute and 45° C. for 10 seconds (where a single acquisition, fluorescent data acquisition, was performed).

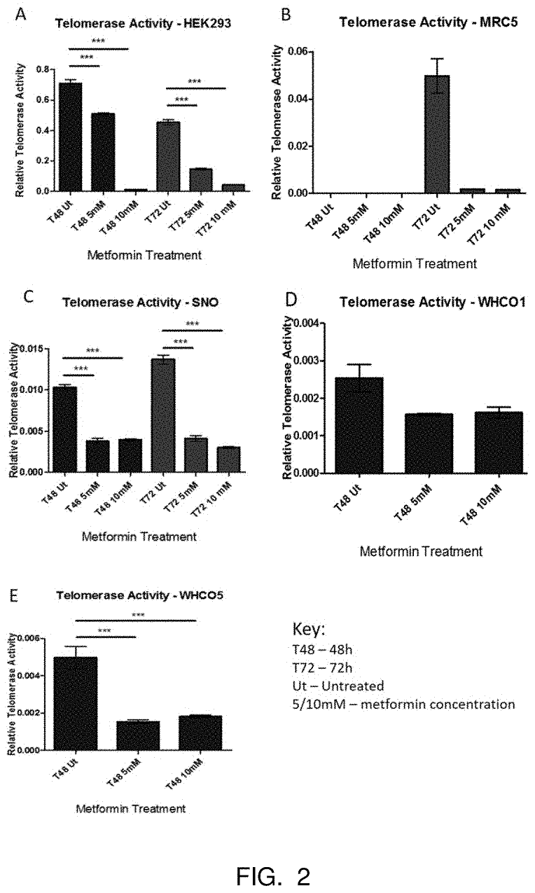

Telomerase activity was measured in all cell lines, using the HEK293 cells as a positive control and the MRC5 cell line as a negative control. Statistical analysis was performed through GraphPad Prism, using ordinary one-way ANOVA at 95% confidence level to compare the metformin treated and untreated samples. Only the 48 h readings were obtained for both the WHCO1 and WHCOS cell lines, as no amplification took place at the 72 h timepoint (including the untreated control). A significant reduction in telomerase activity was seen in the HEK293 (positive control), SNO and WHCOS cell lines (FIG. 2, panels A, C, E) with a downward trend seen in the WHCO1 cell line (FIG. 2, panel D). The MRC5 cell line (FIG. 2, panel B) showed little to no telomerase activity, which was expected from a negative control, however the 72 h untreated MRC5 sample seems to be an outlier.

### Example 3

Nanoparticle Synthesis and Characterisation

Nanoparticle Synthesis Procedure

A concentrated 3:1 mixture of HCl:HNO3 was used to treat the glassware. This prevented nanoparticles (NPs) from attaching themselves to the sides of the glassware. Silver nitrate, chloroplatinic acid and chloroauric acid were reduced using sodium citrate to synthesise silver, platinum and gold nanoparticles, respectively. Deionised water (50 ml) was added to a treated glass beaker. Metal salt was then added to the beaker to a concentration of 1 mM. The beaker was then placed on a hotplate. The solution was stirred and allowed to heat up to 65° C. while being stirred before adding the sodium citrate reducing agent. Temperature is a vital factor in nanoparticle synthesis, so multiple were used to find the optimum temperature for each metal salt. Once the solution reached the desired temperature of 65° C., sodium citrate was added until a colour change occurred. The amount and method of addition of the reducing agent also affects the formation of the NPs so multiple concentrations, and methods (ie. dropwise or rapid addition) were tested. The presence of a crimson colour indicated the formation of gold nanoparticles (AuNPs) within the desired size range (nanoparticle diameter of approximately 13 nm), whereas brown and dark yellow indicated the synthesis of silver and platinum NPs respectively. The solution was then removed from the heat source and allowed to settle for a further 20 minutes with gentle stirring. The size and morphology was then verified using both scanning and transmission electron microscopy (SEM and TEM) as well as UV-visual spectrophotometry and Zetasizer measurements (light scattering). The NPs were then stored at room temperature in the dark.

Electron Microscopy (SEM and TEM)

Scanning electron microscopy (FEI Quanta FEG-SEM) and transmission electron microscopy (FEI Spirit 120 kV TEM) were used to determine the shape and size of the synthesised gold, platinum and silver nanoparticles. Similarly, the lysozyme-conjugated platinum nanoparticles as well as thiol-DNA conjugated gold nanoparticles were viewed under SEM and TEM. Sample preparation for the SEM included adding liquid nanoparticle solution to a carbon film attached to a sample stub. The sample was then dried to ensure that no liquid remains, as this could adversely affect the vacuum within the microscope. Sample preparation for the TEM included adding liquid nanoparticle solution to a 3 mm diameter copper grid coated with lacey carbon. The sample was then dried under a heat lamp until no moisture remained. The nanoparticles would then remain trapped on the lacey carbon.

Size and morphology of the nanoparticle were determined using UV-Vis spectrophotometry, SEM and TEM. ImageJ analysis was performed on the TEM images to determine the average diameter of the spherical nanoparticles. In all three cases, synthesis was successful, however only the gold nanoparticles proved to be of the correct size and uniform morphology needed for most downstream applications.

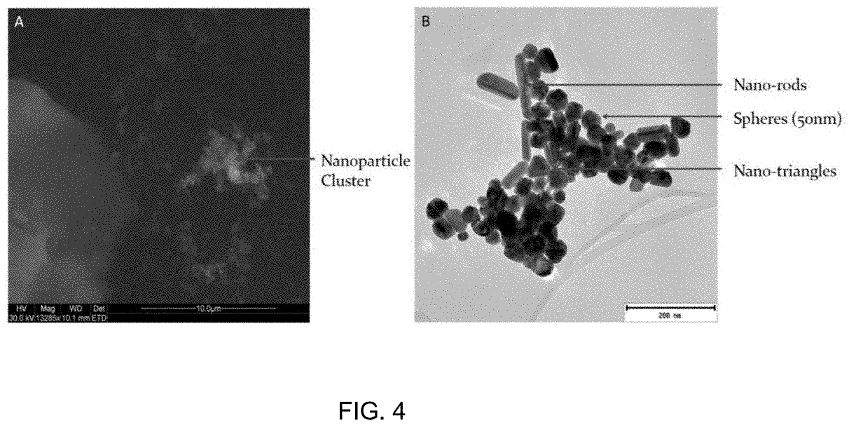

Silver nanoparticles were successfully synthesised and produced a solution with a brown colour. The spectrophotometry results show a large size range for the silver nanoparticles, as indicated by the wide peak (FIG. 3). This was further confirmed by the TEM results (FIG. 4), where nano-rods and nano-triangles of varying sizes were present alongside the 50 nm diameter spherical nanoparticles. This large range in particle shapes and sizes would indicate that these silver nanoparticle solutions are not suitable for downstream applications.

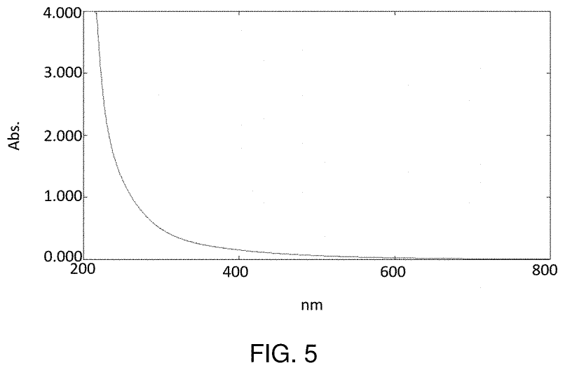

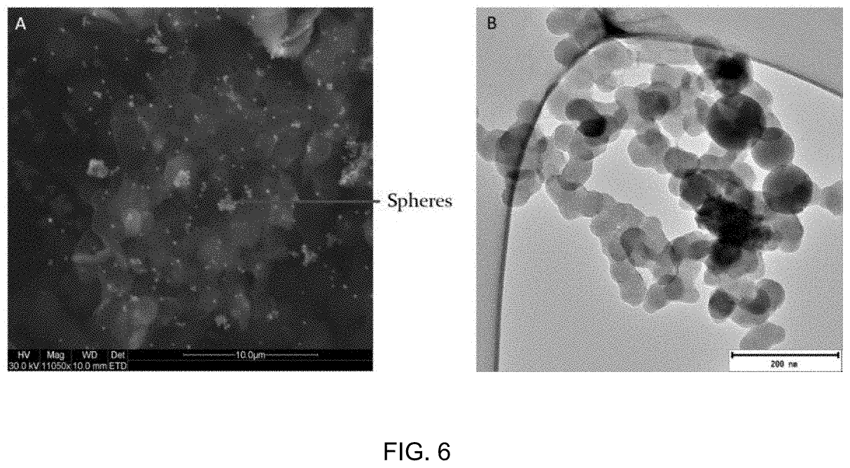

Platinum nanoparticles were synthesised, producing a solution with a dark yellow colour. The Spectrophotogram (FIG. 5) showed no distinct peak, making it difficult to determine the uniformity of the platinum nanoparticles. Indeed, it was thought that synthesis was not successful, however SEM and TEM images (FIG. 6) confirmed the presence of nanoparticles. The TEM image (FIG. 6, panel B) showed that the particles were very closely associated and therefore a more accurate ImageJ analysis was not possible.

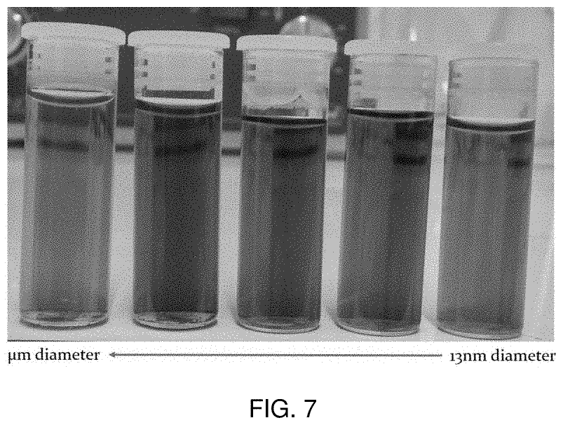

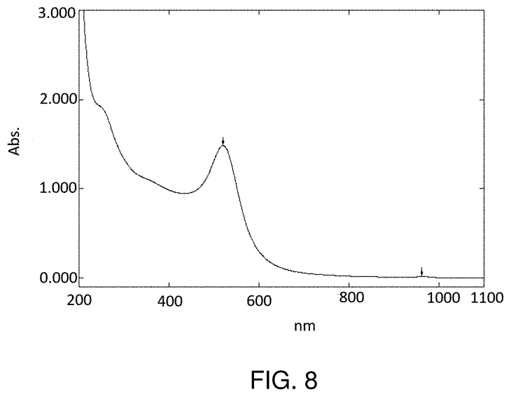

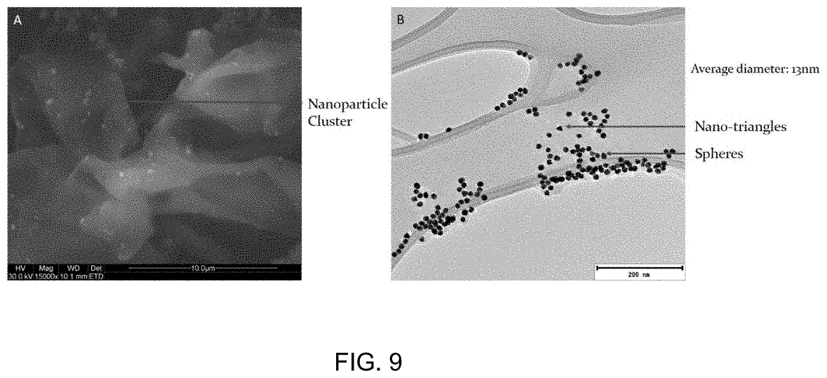

AuNPs were successfully synthesised and produced a solution with a distinct red colour. The size of AuNPs was also very easy to control, with distinct colour changes accompanying the change in size (FIG. 7). If the reducing agent was added slowly to the synthesis reaction at 65° C., the colour of the solution turned from red to purple and then blue. The gold tinge in the far-left vial (FIG. 7) indicates the presence of gold macromolecules/aggregation. The spectrophotometer readings (FIG. 8) showed a distinct thin peak, indicating very uniform particle size distribution. This was also confirmed with the TEM images (FIG. 9, panel B), where after ImageJ analysis it was found that the average diameter of the particles of the red solution was 13 nm. The narrow size distribution as well as the distinct colour differences due to particle size made AuNPs the natural choice for the subsequent DNA functionalisation.

Platinum Nanoparticle Protein Functionalisation

Platinum nanoparticles were first bound to lysozyme as a proof of concept that functionalisation of nanoparticles would result in a change in the UV-visible spectrum, as well as observe any changes under the SEM and TEM. Platinum nanoparticles were selected as they were thought to have a better chance to interact with the amide group found on the protein. The protein was added to 5 ml of diluted platinum nanoparticles (to a final concentration of 30 ng/μl). The nanoparticle-protein solution was then incubated for 1 h at room temperature while being stirred.

The absorbance of the protein-nanoparticle solution was quantified by spectrophotometry (UV-1800 Spectrophotometer (Shimadzu)) and the size and shape of particles were verified by SEM and TEM.

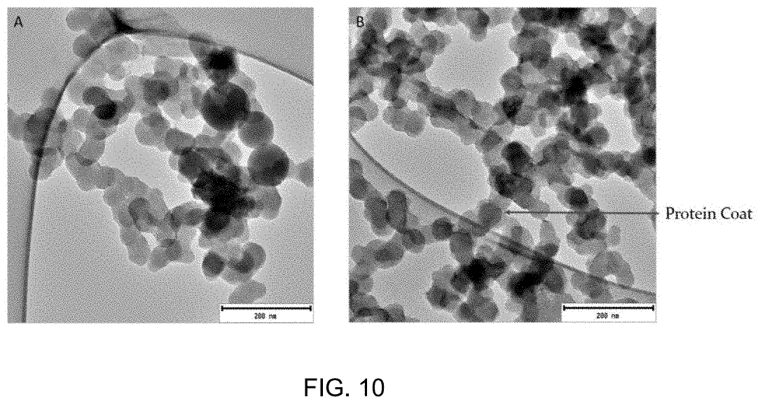

The unmodified platinum nanoparticles looked very similar to the lysozyme modified particles, however a possible protein coating was observed (FIG. 10). Due to the difficulty of viewing platinum nanoparticles under TEM, as well as the lack of a colour change, it was decided to continue the experiment with AuNPs.

AuNP DNA Functionalisation

AuNPs were used for subsequent experiments, due to their easily visible colour change in solution as well as their uniformity. All glassware was treated with 12M NaOH and washed with deionised water to prevent AuNPs from attaching to the glass, as this may affect the yield of the functionalised nanoparticles. Thiolated DNA having the sequence 5′-TTTTTTTTTTAATCCGTCGAGCAGAGTT-3′ (SEQ ID NO:5) or thiolated DNA having the human codon-optimised sequence 5′-TTTTTTTTTTAATCCCAATCCCAATCCCAATCCC-3′ (SEQ ID NO:6) (as 1 mM: 5′-HS(CH2)6TTTTTTTTTTAATCCGTCGAGCAGAGTT-3′; or as 1 mM: 5′-HS(CH2)6TTTTTTTTTTAATCCCAATCCCAATCCCAATCCC-3′) was bound to both gold and platinum nanoparticles using a pH dependant sodium citrate method alongside a simple incubation. This was to improve on existing methods where the functionalisation reaction is labour intensive and time consuming, requiring constant adjustment and taking up to 40h to complete. In addition, a pH dependent, or pH controlled, method for functionalisation of the thiolated DNA to the AuNPs was employed. The pH dependent method relies on the interaction of the thiol-modified DNA with the AuNPs by decreasing the pH of the solution. The pH of the solution is reduced from pH7 to pH2-4. This is achieved using a sodium citrate-hydrochloric acid buffer (pH2).

For the sodium citrate method, the thiolated DNA was added to the nanoparticle solution to a final concentration of 2 μM and was mixed by inversion. The pH-buffered sodium citrate solution was then rapidly added to the mixture to a final concentration of 10 mM and was incubated for 20 minutes at room temperature. For the simple incubation method, the nanoparticle solution was once again brought to a final concentration of 2 μM of thiolated DNA. The solution was then mixed by inversion for 16h to allow the nanoparticles to attach to the thiolated DNA. The absorbance of the functionalised particles was read on the UV-vis spectrophotometer along with a control solution containing no DNA. The DNA modified AuNPs were then centrifuged at 13000 rpm for 45 minutes to remove any excess DNA. The modified AuNPs were then re-suspended in deionised water.

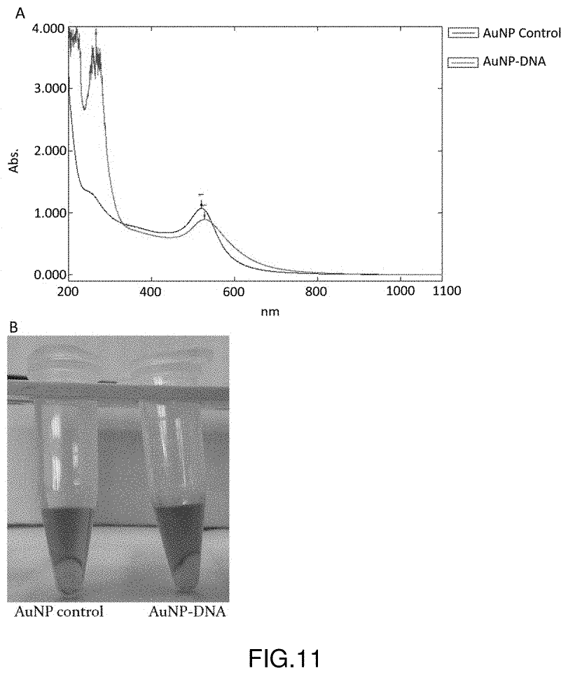

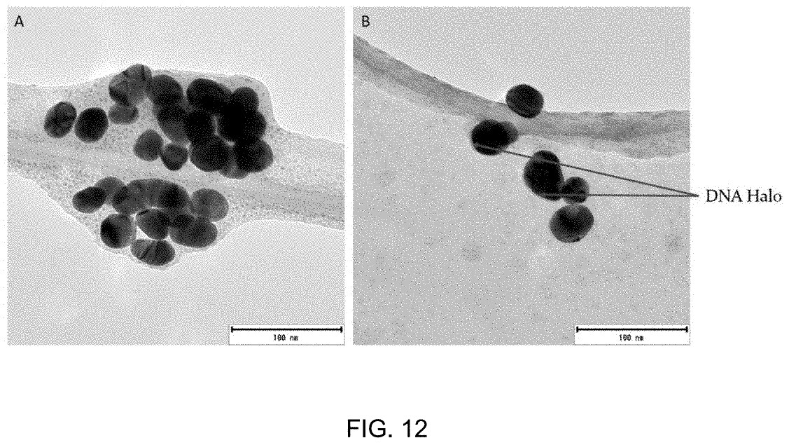

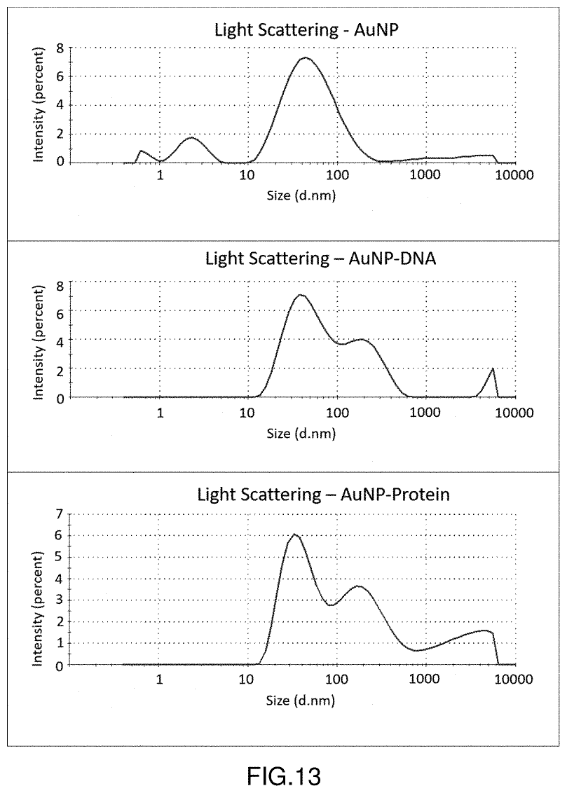

The simple 16h incubation method was also tested, as the sodium citrate buffer method caused rapid aggregation after which the nanoparticles would not re-disperse. DNA coupled AuNPs were then characterised using TEM as well as spectrophotometry. The spectrophotometer results (FIG. 11, panel A) showed a distinct shift after functionalisation. The lowering of the peak could indicate that the particles are more closely associated. This shift is consistent with an increase in size of the modified nanoparticles. Panel B of FIG. 11 shows the colour change that occurred due to the functionalisation. This blue shift in colour is once again consistent with larger (or more closely associated) AuNPs. TEM images (FIG. 12) show a distinct halo around modified AuNPs (panel B), which cannot be seen in the unmodified particles (panel A). This halo seems to indicate the presence of DNA on the surface of the AuNPs. Using a Zetasizer (FIG. 13), the size of the nanoparticles could be determined using light scattering. Panel A of FIG. 13 shows a 40 nm peak suggesting that the average particle size of the AuNP control is 40 nm in diameter. The DNA functionalised sample (FIG. 13, panel B) shows two peaks at 40 and 110 nm, suggesting that there are two particle sizes predominantly found in the solution this may be due to some unfunctionalised particles. The sample containing the DNA functionalised AuNPs and the HEK293 protein extract (FIG. 13, panel C) shows the two peak sizes (at 40 nm and 110 nm) seen in panel B, however there is evidence of massive aggregation (peak from 1000 nm to 6000 nm).

In an attempt to improve on the speed and yield of the functionalization methods, a pH dependent method is proposed. For the pH dependent method of functionalization, thiol-DNA was added to 5 ml of the nanoparticle solution to a final concentration of 2 μM. The solution was mixed to ensure an even distribution of the thiol-DNA. Once mixed, the low pH sodium citrate-hydrochloric acid buffer (pH2) was added drop-wise until the pH of the solution was decreased to below pH4. AuNPs synthesised with sodium citrate are often surrounded by a negative charge which decreases the ability of the thiol-DNA to bind. The decreased pH of the solution results in the thiolated DNA binding more readily to the AuNPs and increases binding efficiency. This pH dependent method of functionalizing DNA to the AuNPs takes approximately 5-10 minutes to achieve binding of most of the free thiol-DNA to the gold nanoparticles. The DNA-modified AuNPs were then centrifuged at high rpm for 5 minutes to purify them. No centrifugation step after the synthesis of the gold nanoparticles and prior to functionalisation was required.

### Example 4

Nanoparticle Telomerase Activity Assay

AuNP Based Telomerase Activity Assay

The AuNP based telomerase activity assay was designed as a PCR reaction. A buffer was added to the DNA functionalised nanoparticle solution containing essential components needed by the telomerase enzyme to elongate the telomeric DNA substrate (20 mM Tris-HCL at pH8.3, 6.3 mM KCL, 1 mM EGTA, 0.005% Tween-20, 0.1 mg/ml BSA, 1.5 mM MgCl2, 1 mM dNTPs). Protein extract (extracted using CHAPS lysis buffer as described in Example 2), from HEK293 cells, was then added to the solution to a final concentration of 20 ng/μl, to keep it consistent with the qPCR telomerase activity assay. The solution was then incubated at 45° C. for up to 2h to ensure that the DNA was significantly elongated and to keep the temperature in line with the temperatures used in the qPCR based telomeres activity assay. After the extension reaction, the solution was transferred directly to a 96-well plate and the absorbance was read in an ELISA reader at 530 nm. This wavelength was chosen as it was determined to be the peak absorbance for the AuNP solution used in this experiment. The assay was then performed on SNO and WHCOS cell lines due to the results obtained from the TRAPeze kit. Changes in the absorbance of the samples were then compared to the results obtained from the qPCR telomerase activity assay. A decrease in absorbance should indicate a decreased telomerase activity, as the nanoparticles associate closer together. This could then be compared to the decrease found with the TRAPeze qPCR kit.

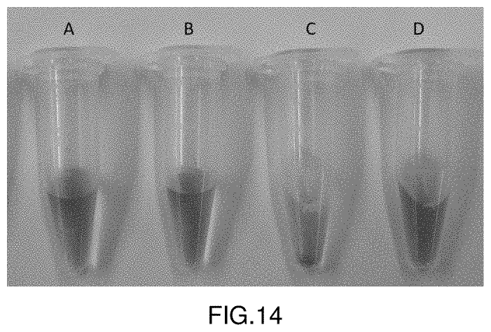

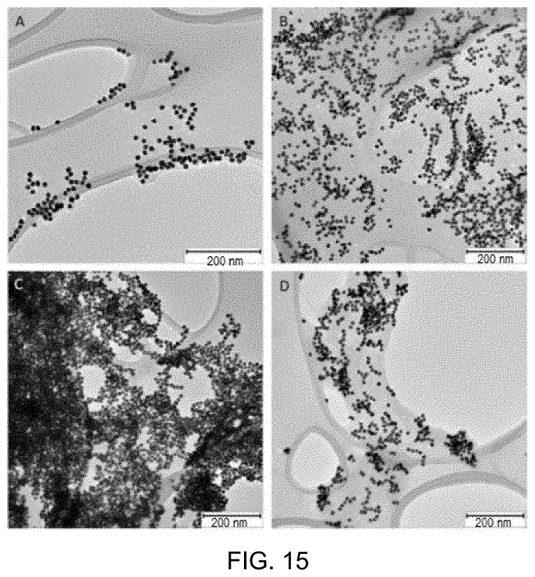

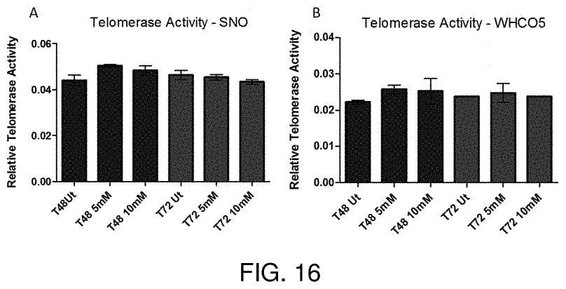

The colour change was measured using a spectrophotometer. Thereafter the solutions were viewed under TEM. Although a distinct colour change was seen compared to a negative control (FIG. 14), the sample that was allowed to elongate the attached DNA did not show as much aggregation as in the negative control. This was further confirmed by TEM images (FIG. 15), where the negative control showed massive aggregation compared to the sample that was allowed to elongate the attached telomeric DNA. This observed difference could signify telomerase activity. The particles in containing DNA modified AuNPs and reaction buffer showed slightly more association, which could account for its more purple colour (FIG. 14). After performing the reaction, absorbance readings did not find any significant difference between metformin treated and untreated samples (FIG. 16) although a difference could previously be seen between elongated and non-elongated samples.

### Example 5

Telomerase Activity Assay Using Gold Nanoparticles Functionalised with Thiolated Linker

Synthesis of AuNPs for Linker Functionalisation

Gold nanoparticles (AuNPs) coupled to a 3′ thiolated linker nucleic acid strand which contains a complimentary sequence for an extension nucleic acid strand having a telomerase recognition site that binds telomerase were also synthesised. AuNPs were synthesized using the chemical hydrothermal method, where a rapidly stirring 50 ml solution of 0.5 mM gold chloride is first brought to a boil before 2 ml of 10 mg/ml sodium citrate is rapidly added. The reaction completes after 5 minutes to form AuNPs with a diameter of 13-16 nm. The AuNP solution was filter sterilized and kept in 0.02% sodium azide to prevent contamination. The AuNPs were then characterized using UV-Vis spectrophotometry and transmission electron microscopy (TEM) as described in the previous examples.

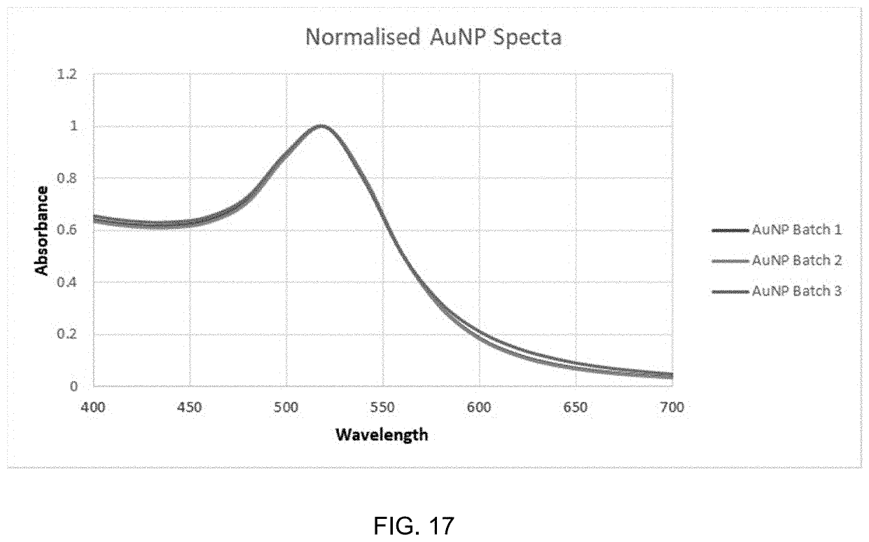

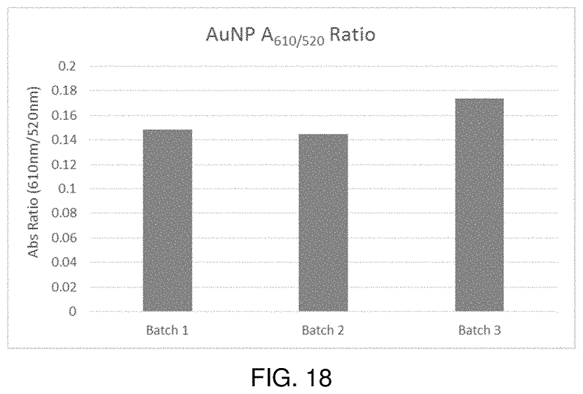

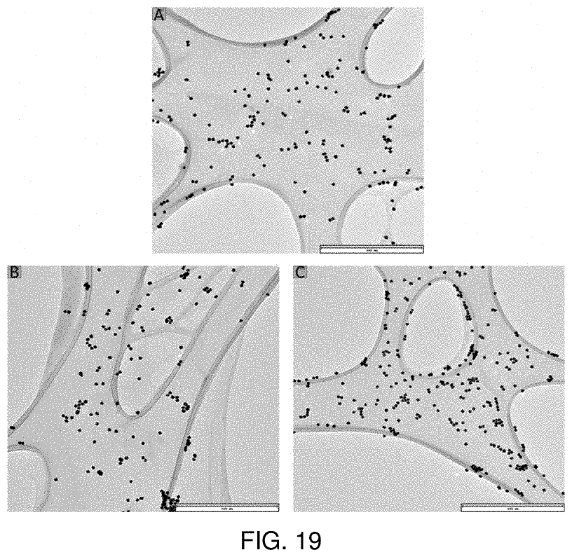

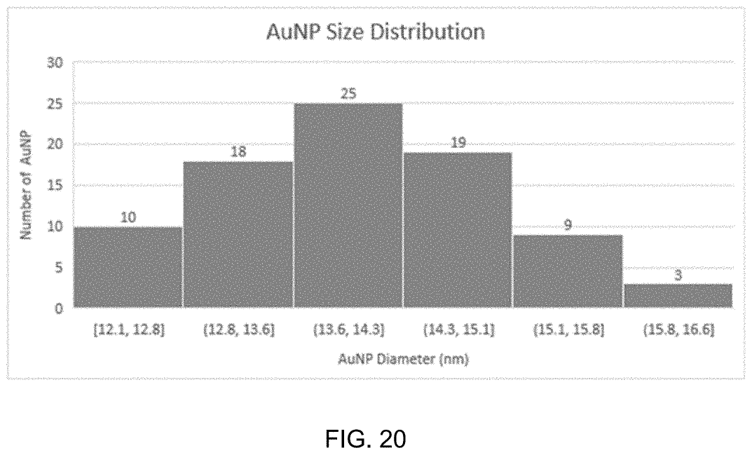

The UV-Vis spectra (400 nm-700 nm) obtained for three separate batches of AuNP solutions show that all three spectra very closely align. Thus the synthesis method was consistent and repeatable (FIG. 17). Further, the ratio of the absorbance at two wavelengths (610 nm and 520 nm) of three batches of AuNPs was determined. Using a ratio instead of a simple absorbance reading provides more reliable and accurate results. The A610/520 ratios of the three batches are very similar, indicating that the AuNP synthesis method is consistent and reproducible (FIG. 18). Finally, the TEM images of each of the three batches were compared and the particles appeared to be of similar size and spherical in shape, providing further evidence of the consistency and repeatability of this method (FIG. 19). Based on the TEM images, the size distribution of the AuNPs was determined. The diameter of the spherical nanoparticles was obtained using ImageJ. The average diameter was found to be 14 nm with a standard deviation of 0.936. The size of these AuNPs is ideal for downstream applications, specifically a telomerase activity assay (FIG. 20).

pH Dependent Functionalisation of AuNPs with Linker

For functionalization of AuNPs with the linker nucleic acid, 90 μl of 0.1 mM 3′ thiolated-DNA having the nucleotide sequence of SEQ ID NO:7 was added to 3 ml of sterile AuNP solution and mixed for 2 minutes. Thereafter, 100 μl of 10 mM sodium citrate (pH 1) was added to reduce the overall pH of the solution to pH 3. The solution was gently stirred for 10 minutes before centrifugation at 6000 g for 10 minutes. The supernatant was discarded, and the pellet was resuspended in dH₂O.

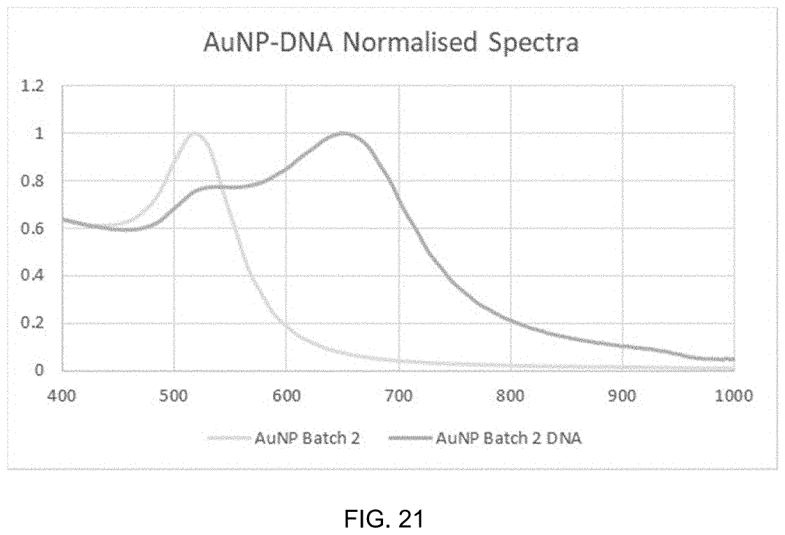

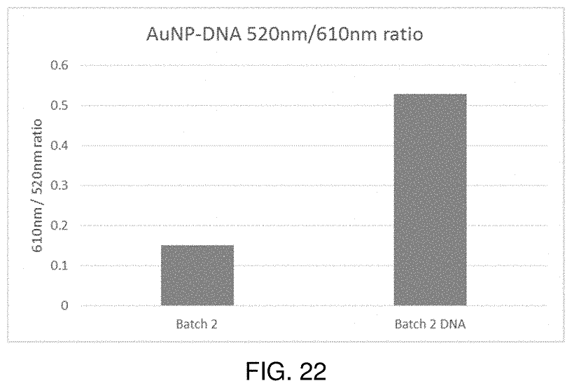

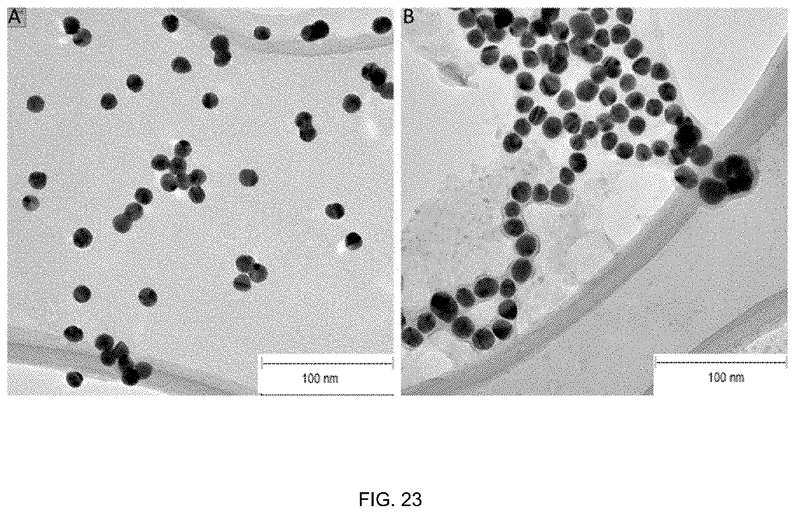

The solution was then characterized using TEM and UV-Vis spectrophotometry. UV-Vis spectrophotometry of the thiol-DNA functionalised AuNPs showed that the unmodified AuNPs have a peak at 520 nm and the linker nucleic acid functionalised AuNPs have a peak at 650 nm as well as a smaller peak at 520 nm. These spectra represent a large shift in the absorbance after the addition of the linker thiol-DNA to the surface of the AuNPs, indicating that most of the AuNPs were surface modified with the linker nucleic acid (FIG. 21). The ratio of the absorbance at two wavelengths (610 nm and 520 nm) of both unfunctionalised and functionalised AuNPs was determined. Using a ratio instead of a simple absorbance reading gives more reliable and accurate results. The addition of the linker DNA to the surface of the AuNPs caused the ratio to increase (FIG. 22). Further, TEM analysis showed that there is a distinct halo around many of the linker DNA functionalised AuNPs which was not observed for the unmodified particles. This indicates the presence of linker DNA on the surface of the AuNPs (FIG. 23).

Linker-Functionalised AuNPs and Telomerase Activity Assay

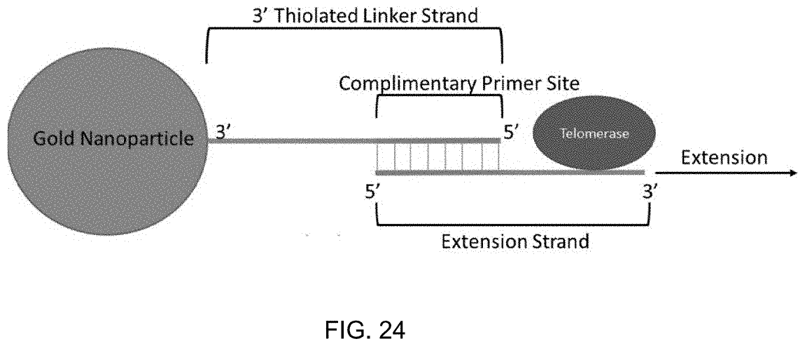

The nucleic acid linker coupled to the AuNPs contains a complimentary sequence for an extension nucleic acid strand (SEQ ID NO:8). The extension nucleic acid strand contains a telomerase recognition site that binds telomerase and in this way the extension strand is elongated by telomerase in a telomerase activity assay (FIG. 24). This elongation can be detected using UV-Vis spectrophotometry. Compared to a single oligonucleotide setup as described in the previous examples, where the gold nanoparticle is functionalised with the thiolated telomerase substrate itself, the linker-functionalised AuNPs are more stable and aggregate less.

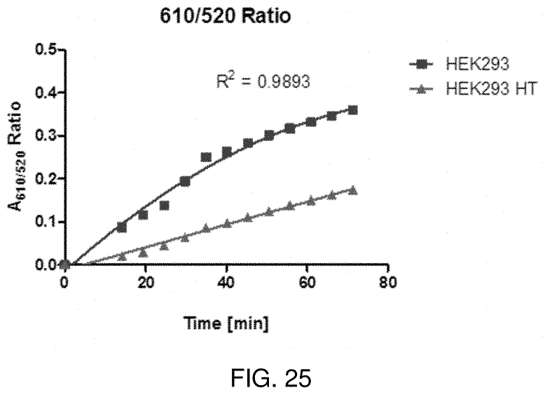

The linker nucleic acid AuNP based telomerase activity assay was performed as described in Example 4 on HEK293 cells and HEK293 heat treated cells (with the telomerase inactivated) as a control. After the extension reaction, the absorbance was read at 610 nm and 520 nm and a absorbance ratio was obtained (A_{610/520}). The HEK293 telomerase positive control showed higher ratio than that of the heat-treated (telomerase inactivated) control as was expected (FIG. 25).

The linker-functionalised AuNPs also have the potential to be reused, as the extension nucleic acid strand can be decoupled from the linker nucleic acid strand by simple thermal denaturation. The elongated extension nucleic acid strand can then be removed for downstream use and a new extension nucleic acid strand can be added to the thiol-DNA modified AuNPs. The elongated extension nucleic acid strand can further be amplified by PCR, using a forward primer of SEQ ID NO:9, and resolved on a gel to generate a single telomere length analysis (STELA)-like pattern. This not only allows for a simple colorimetric telomerase activity assay, but also a quantitative STELA-like analysis (FIG. 26). Table 2 shows the sequences of the thiolated linker nucleic acid, the extension nucleic acid and the forward PCR primer for use in amplifying the extension nucleic acid for quantitative analysis.

TABLE 2
Nucleic acid sequences for the linker-
functionalised AuNP telomerase activity assay.

| Name | Sequence 5′→ 3′ | SEQ ID NO |
|---|---|---|
| Linker nucleic | CATGTGTTTCGTTAG | SEQ ID NO: 7 |
| acid | GCACCTAAGGCTAGC | |
| | TTTTTTTTTT | |
| Extension nucleic | CTTAGGTGCCTAACG | SEQ ID NO: 8 |
| acid | AAACACATGAATCCG | |
| | AATCCGAATCCGAAT | |
| | CCGAATCCG | |
| Extension nucleic | GAATCCACGGATTGC | SEQ ID NO: 9 |
| acid PCR Primer | TTTGTGTAC | |

Bold denotes regions of complementarity of the sequences.

### REFERENCES

Bey, E., Alexander, J., Whitcutt, J. M., Hunt, J. A., and Gear, J. H. S. (1976). Carcinoma of the esophagus in africans: Establishment of a continuously growing cell line from a tumor specimen. In Vitro 12, 107-114.
Graham, F. L., Smiley, J., Russell, W. C., and Nairn, R. (1977). Characteristics of a human cell line transformed by DNA from human adenovirus type 5. J. Journal of General Virology. 36, 59-74.
Jacobs, J. P., Jones, C. M., and Bailie, J. P. (1970). Characteristics of a Human Diploid Cell Designated MRC-5. Nature. 227, 168-170.
Veale, R. B., and Thomley, A. L. (1989). Increased Single Class Low-Affmity EGF Receptors Expressed by Human Oesophageal Squamous Carcinoma Cell Lines. South African Journal of Science. 85, 375-379.

1. A method of producing gold nanoparticles functionalised to detect telomerase activity, the method comprising:
a) combining a gold salt with sodium citrate to obtain a gold nanoparticle solution;
b) adding a thiolated nucleic acid to the gold nanoparticle solution; and
c) adding a buffer having a pH of about 1-4 to obtain a functionalised gold nanoparticle solution;
wherein the thiolated nucleic acid is (i) a thiolated telomerase substrate; or (ii) a thiolated linker nucleic acid having complementarity to a nucleic acid comprising a telomerase substrate.

2. The method of claim 1, further comprising centrifuging the functionalised gold nanoparticle solution obtained in (c) and recovering the functionalised gold nanoparticles.

3. The method of claim 1, wherein no centrifugation is performed between (a) and (b).

4. The method of claim 1, wherein the thiolated nucleic acid has a sequence corresponding to SEQ ID NO:5, SEQ ID NO:6, or SEQ ID NO:7.

5. The method of claim 1, wherein the buffer has a pH of about 1 or about 2 and wherein the pH of the functionalised gold nanoparticle solution after (c) is about 2 to 4.

6. The method of claim 5, where in the pH of the functionalised gold nanoparticle solution after (c) is about 3.

7. The method of claim 1, further comprising adding a telomerase substrate having a region of complementarity to the thiolated linker nucleic acid.

8. A functionalised gold nanoparticle solution obtained by the method of claim 1.

9. A functionalised gold nanoparticle produced by the method of claim 1.

10. A functionalised gold nanoparticle complex, comprising:
(a) a gold nanoparticle coupled to a linker nucleic acid having complementarity to a nucleic acid comprising a telomerase substrate; and
(b) a nucleic acid comprising a telomerase substrate,
wherein the nucleic acid comprising the telomerase substrate is hybridised to the linker nucleic acid to form the functionalised gold nanoparticle complex.

11. The functionalised gold nanoparticle complex of claim 10, wherein the linker nucleic acid has a sequence corresponding to SEQ ID NO:7.

12. The functionalised gold nanoparticle complex of claim 10, wherein the nucleic acid comprising the telomerase substrate has a sequence corresponding to SEQ ID NO:8.

13. A telomerase assay for detecting telomerase activity in a cell, comprising detecting telomerase activity in the cell using the functionalised gold nanoparticle of claim 9, or a functionalised gold nanoparticle complex comprising:
(a) a gold nanoparticle coupled to a linker nucleic acid having complementarity to a nucleic acid comprising a telomerase substrate; and
(b) a nucleic acid comprising a telomerase substrate,
wherein the nucleic acid comprising the telomerase substrate is hybridised to the linker nucleic acid to form the functionalised gold nanoparticle complex.

14. The telomerase assay of claim 13 wherein the detecting comprises detecting a colorimetric change in the cell or a sample of cells when telomerase is present compared with a reference cell or sample of cells.

15. The telomerase assay of claim 14, wherein the colorimetric change is detected using spectrophotometry.

16. The telomerase assay of claim 13, wherein the telomerase activity is detected in vitro.

17. The telomerase assay of claim 13, wherein the telomerase activity is detected in vivo in a subject.

18. The telomerase assay of claim 13, further comprising assessing telomerase modulating ability of a compound by comparing (i) the telomerase activity when the compound is present in the cell with (ii) the telomerase activity when the compound is not present in the cell.

19. The telomerase assay of claim 13, wherein the cell is a proliferative cell.

20. The telomerase assay of claim 19, wherein the proliferative cell is a cancer cell, and skin cell, a hair follicle cell, and/or a blood cell.

21. A kit including the functionalised gold nanoparticle of claim 9 or a functionalised gold nanoparticle complex comprising:
(a) a gold nanoparticle coupled to a linker nucleic acid having complementarity to a nucleic acid comprising a telomerase substrate; and
(b) a nucleic acid comprising a telomerase substrate,
wherein the nucleic acid comprising the telomerase substrate is hybridised to the linker nucleic acid to form the functionalised gold nanoparticle complex.
